(19) United States
(12) Patent Application Publication
(10) Pub. No.: US 2003/0232078 A1
(43) Pub. Date: Dec. 18, 2003

(54) Formulation & dosage form for the controlled delivery of therapeutic agents

(76) Inventors: Liang C. Dong, Sunnyvale, CA (US); Patrick S. L. Wong, Burlingame, CA (US); Steve Espinal, Mountain View, CA (US); Vu A. Nguyen, San Jose, CA (US)

Correspondence Address: ALZA CORPORATION, P O BOX 7210, INTELLECTUAL PROPERTY DEPARTMENT, MOUNTAIN VIEW, CA, 940397210

(21) Appl. No.: 10/324,239

(22) Filed: Dec. 19, 2002

Related U.S. Application Data
(60) Provisional application No. 60/343,001, filed on Dec. 19, 2001.

(51) Int. Cl. A61K 31/522; A61K 31/198; A61K 9/22; A61K 31/155

(52) U.S. Cl. 424/468; 514/263.36; 514/567; 514/635

(57) ABSTRACT

The present invention includes a formulation and controlled release dosage form that enable the controlled release of therapeutic agents showing reduced absorption in the lower gastrointestinal tract. The formulation of the present invention includes a therapeutic agent that exhibits greater absorption in the upper GI tract than in the lower GI tract and a permeation enhancer, which serves to increase absorption of the therapeutic agent in the lower GI tract. The formulation of the present invention further includes a carrier that allows the formulation to transition to a bioadhesive gel in-situ after the formulation is dispensed within the GI tract and has had some opportunity to reach the surface of the GI mucosal membrane. The bioadhesive gel formed by the formulation of the present invention works to present effective concentrations of both the therapeutic agent and the permeation enhancer at the surface of the GI mucosal membrane over a period. The controlled release dosage form of the present invention is designed to deliver the formulation of the present invention at a desired release rate or release rate profile over a desired period of time.

### RELATED CASES

This is a non-provisional application claiming priority under 35 U.S.C. §119 from U.S. provisional application No. 60/343,001, filed on Dec. 19, 2001, the contents of which are incorporated by reference.

### BACKGROUND

1. Field of the Invention

The present invention relates to formulations and dosage forms that enable the controlled release of therapeutic agents that are poorly absorbed in the lower gastrointestinal (GI) tract. In particular, the present invention relates to in-situ gelling formulations and dosage forms for delivering such formulations that facilitate the controlled release of therapeutic agents are characterized by limited absorption or no absorption in the lower GI tract.

2. State of the Art

Dosage forms providing the controlled-release of therapeutic agent formulations within the GI tract are known in the art. U.S. Pat. Nos. 4,627,850, 5,234,280, 5,413,572, and 6,174,547 teach various exemplary controlled-release capsules that allow controlled delivery of liquid drug formulations. It is generally appreciated that controlled release dosage forms may provide advantages relative to dosage forms that are incapable of controlled delivery of therapeutic agents. For example, controlled release dosage forms generally allow more precise control of therapeutic agent plasma concentrations over extended periods of time, and, as a result, controlled release dosage forms may be used to minimize patient compliance issues, reduce undesirable side affects, and increase or achieve a therapeutic benefit provided by the therapeutic agent delivered.

Whether they are designed to deliver liquid or solid formulations, controlled release dosage forms release therapeutic agent at a desired release rate or release rate profile over an extended period of time as the dosage forms pass through the GI tract. Significantly, because of their limited transit time in the upper GI tract, controlled release dosage forms generally release most of the active agent they contain in the lower GI tract, such as in the colon. As a consequence, it is generally the case that, in order for a controlled release dosage form to be effective, the therapeutic agent released by the controlled release dosage form must be absorbable through the lower regions of the GI tract. However, the GI absorption of various therapeutic agents, which may otherwise benefit from controlled release of a period of time, is limited to the upper GI tract (e.g., the stomach and small intestine). For example, it is well known that acyclovir, gancyclovir, L-dopa, carbidopa, and ABT-232 are not absorbed in significant amounts through the mucosal membrane of the lower GI tract, and when such therapeutic agents are delivered in the lower GI tract using conventional formulations, the bioavailability of these therapeutic agents is dramatically reduced. Therefore, conventional controlled release dosage forms and formulations are not well suited for the controlled delivery of therapeutic agents that are less readily absorbed in the lower GI tract than in the upper GI tract.

It would be an improvement in the art to provide a formulation and dosage form that enable the controlled release of therapeutic agents that exhibit decreased absorption in the lower GI tract relative to the upper GI tract. In order to be effective, such a formulation and dosage form must not only facilitate controlled release of the active agent over a desired period of time, but the formulation and dosage form should also enhance the bioavailability of the therapeutic agent in the lower GI tract. As is easily appreciated, a formulation and dosage form allowing the controlled release of therapeutic agents having reduced absorption in the lower GI tract could bring the benefits of controlled delivery to orally administered therapeutic agents that may not now be practically administered using conventional controlled release dosage forms.

### SUMMARY OF THE INVENTION

The present invention includes a formulation that allows the controlled release of therapeutic agents that exhibit reduced absorption in the lower GI tract. The formulation of the present invention includes a therapeutic agent having a relatively higher absorption in the upper GI tract than in the lower GI tract and a permeation enhancer, which serves to increase absorption of the therapeutic agent in the lower GI tract. In order for a permeation enhancer to successfully increase the absorption of a therapeutic agent across the GI mucosal membrane of the lower GI tract, however, the concentration of the permeation enhancer must be maintained above a critical level at the surface of the GI mucosal membrane. Therefore, the formulation of the present invention further includes a carrier that allows the formulation to transition to a bioadhesive gel in-situ after the formulation is dispensed within the GI tract and has had some opportunity to reach the surface of the GI mucosal membrane. The bioadhesive gel formed by the formulation of the present invention works to present effective concentrations (i.e., concentrations sufficient to increase absorption of the therapeutic agent through the mucosal membrane of the lower GI tract) of both the therapeutic agent and the permeation enhancer at the surface of the GI mucosal membrane over a period of time sufficient to enhance absorption of the therapeutic agent in the lower GI tract.

The present invention further includes a controlled release dosage form incorporating the formulation of the present invention. The dosage form of the present invention may include any controlled release delivery device capable of delivering the formulation of the present invention within the GI tract of an intended subject at a desired release rate or release rate profile. For example, the dosage form of the present invention may deliver the formulation of the present invention at a zero order, ascending, descending, or pulsatile release rate over a desired period of time within the GI tract. Because the formulation delivered by the dosage form of the present invention enhances the absorption of the therapeutic agent in the lower GI tract, the dosage form of the present invention facilitates the controlled release of therapeutic agents that may not otherwise feasibly delivered in a controlled manner from an oral dosage form.

### BRIEF DESCRIPTION OF THE DRAWINGS

FIG. 1 through FIG. 5 illustrate various views of controlled release dosage forms of the present invention fabricated using hard gelatin capsules.

FIG. 6 and FIG. 7 provide exterior and cross-sectional views of a controlled release dosage form according to the present invention fabricated using a soft gelatin capsule.

FIG. 8 and FIG. 9 provide exterior and cross-sectional views of the controlled release dosage form illustrated in FIG. 6 and FIG. 7 during operation.

FIG. 10 and FIG. 11 illustrate a second controlled release dosage form according to the present invention fabricated using a soft gelatin capsule.

FIG. 12 and FIG. 13 illustrate a third controlled release dosage form according to the present invention fabricated using a soft gelatin capsule.

FIG. 14A through FIG. 14D illustrate a method for forming a sealed exit orifice for a controlled release dosage form of the present invention fabricated using a soft gelatin capsule.

FIG. 15 and FIG. 16 illustrate a controlled release dosage form according to the present invention having a sealed exit orifice fabricated as shown in FIG. 14A through FIG. 14D.

FIG. 17 through FIG. 19 illustrate a second method for forming a sealed exit orifice for a controlled release dosage form of the present invention fabricated using a soft gelatin capsule.

FIG. 20 provides a graph illustrating the viscosity of Cremophor EL (ethoxylated castor oil), an exemplary carrier, as a function of water content.

FIG. 21 provides a graph showing the G′ (storage modulus), G″ (loss modulus), and δ (G″/G′) of various Cremophor EL/water blends as a function of water content.

FIG. 22 provides a graph illustrating the dynamic viscosity of various Cremophor EL/water blends.

FIG. 23 provides a graph illustrating the adhesion of various Cremophor EL/water blends.

FIG. 24 provides a schematic representation of a dosage form according to the present invention.

FIG. 25 provides a graph illustrating the release profile of acyclovir delivered at a controlled rate via a dosage form according to the present invention.

FIG. 26 provides a graph describing the plasma concentration profile and bioavailability of acyclovir delivered from a dosage form of the present invention.

### DETAILED DESCRIPTION OF THE INVENTION

The formulation of the present invention includes a therapeutic agent, a permeation enhancer, and a carrier that exhibits in-situ gelling properties. The formulation of the present invention is delivered within the GI tract as a liquid having at least some affinity for the surface of the GI mucosal membrane. Once released within the GI tract, however, it is believed that the formulation of the present invention spreads across one or more areas of the surface of the GI mucosal membrane, where the formulation then transitions into a bioadhesive gel in-situ.

The precise amounts of each component of the formulation of the present invention will vary according to several factors. Among such factors are the particular therapeutic agent to be delivered, the condition to be treated, and the nature of the intended subject. However, in each instance, the amount of each component of the formulation of the present invention is chosen to facilitate increased absorption of the therapeutic agent through the lower GI tract of the subject.

The therapeutic agent included in the formulation of the present invention generally comprises about 0.01 wt % to about 50 wt % of the formulation. As used herein, the term “therapeutic agent” encompasses any entity that may provide a therapeutic benefit to an animal or human subject and exhibits greater absorption in the upper GI tract than in the lower GI tract. Specific therapeutic agents that may be included in the formulation of the present invention include, for example, acyclovir, gancyclovir, L-dopa, carbidopa, ABT-232, and metformin hydrochloride. In addition, the formulation of the present invention may include more than one therapeutic agent. Where more than one therapeutic agent is incorporated into the formulation of the present invention, the combined weight percent of the included therapeutic agents accounts for between about 0.01 wt % and 50 wt % of the formulation.

The specific amount of therapeutic agent included in the formulation of the present invention will vary according to the nature of the therapeutic agent, the dose of therapeutic agent needed to achieve a therapeutic benefit, the dose of formulation administered, and the bioavailability of the therapeutic agent when delivered using the formulation of the present invention. In each instance, however, the formulation of the present invention will include an amount of therapeutic agent sufficient to create and maintain a concentration gradient across the GI mucosal membrane that enables increased absorption of the therapeutic agent in the lower GI tract.

The permeation enhancer included in the formulation of the present invention may include any entity that is compatible with the formulation of the present invention and facilitates absorption of the chosen therapeutic agent across the mucosal membrane of the GI tract. Permeation enhancers suitable for use in the formulation of the present invention include, but are not limited to, ethylene-diamine tetra-acetic acid (EDTA), bile salt permeation enhancers, such as sodium deoxycholate, sodium taurocholate, sodium deoxycholate, sodium taurodihydrofusidate, sodium dodecylsulfate, sodium glycocholate, taurocholate, glycocholate, taurocheno-deoxycholate, taurodeoxycholate, deoxycholate, glycodeoxycholate, and ursodeoxycholate, fatty acid permeation enhancers, such as sodium caprate, sodium laurate, sodium caprylate, capric acid, lauric acid, and caprylic acid, acyl carnitines, such as palmitoyl carnitine, stearoyl carnitine, myristoyl carnitine, and lauroyl carnitine, and salicylates, such as sodium salicylate, 5-methoxy salicylate, and methyl salicylate. Permeation enhancers generally function by opening the tight junctions formed between epithelial cells of the GI mucosal membrane, thereby allowing diffusion (i.e., pericellular transport) of therapeutic agent into the GI mucosal membrane. Though the amount of permeation enhancer included in the formulation of the present invention will generally range between about 11 wt % and about 30 wt %, the precise nature and amount of permeation enhancer included in the formulation of the present invention will vary depending on, for example, the anticipated subject, the therapeutic agent to be delivered, the nature of the permeation enhancer itself, and the dose of formulation to be administered.

It has been generally found that the performance of the permeation enhancer is critically dependent upon the concentration of permeation enhancer present at or near the surface of the GI mucosal membrane. Therefore, the amount of permeation enhancer included in the formulation should be sufficient to maintain an effective concentration of permeation enhancer (i.e., a concentration above the critical concentration for the permeation enhancer used) at or near the surface of the GI mucosal membrane over a period of time sufficient to increase absorption of the therapeutic agent in the lower GI tract. Where possible, the permeation enhancer can be chosen such that the permeation enhancer not only facilitates absorption of the chosen therapeutic agent, but also resists dilution by lumenal fluids or secretions.

The carrier of the formulation of the present invention allows the formulation to transition from a relatively non-adhesive, low viscosity liquid to a relatively viscous, bioadhesive gel after the formulation has been delivered within the GI tract of a subject. The carrier of the formulation of the present invention is chosen such that the transition from a relatively non-adhesive, low viscosity liquid to a relatively viscous, bioadhesive gel occurs after the formulation has been released within the GI tract and has had some opportunity to arrive at the surface of the GI mucosal membrane. Hence, the carrier of the formulation of the present invention enables the in-situ transition of the formulation from a liquid to a bioadhesive gel. Due to its high viscosity and bioadhesive properties, the gel formed by the formulation of the present invention holds the permeation enhancer and the therapeutic together at the surface of the GI mucosal membrane. The bioadhesive gel formed by the formulation of the present invention also protects the therapeutic agent and permeation enhancer from dilution by lumenal fluids to an extent that effective concentrations of both the therapeutic agent and permeation enhancer are maintained at the surface of the GI mucosal membrane over a period of time sufficient to increase absorption of the therapeutic agent.

Suitable carriers that exhibit in situ gelling properties include non-ionic surfactants that transition from a relatively non-adhesive, low viscosity liquid to a relatively viscous, bioadhesive liquid crystal state as they absorb water. Specific examples of non-ionic surfactants that may be used as the carrier in the formulation of the present invention include, but are not limited to, Cremophor (e.g., Cremophor EL and Cremophor RH), Incordas 30, polyoxyethylene 5 castor oil, polyethylene 9 castor oil, polyethylene 15 castor oil, d-α-tocopheryl polyethylene glycol succinate (TPGS), monoglycerides, such as myverol, aliphatic alcohol based nonionic surfactants, such as oleth-3, oleth-5, polyoxyl 10 oleyl ether, oleth-20, steareth-2, stearteth-10, steareth-20, ceteareth-20, polyoxyl 20 cetostearyl ether, PPG-5 ceteth-20, and PEG-6 capryl/capric triglyceride, Pluronic® and tetronic block copolymer non-ionic surfactants, such as Pluronic® L10, L31, L35, L42, L43, L44, L62, L61, L63, L72, L81, L101, L121, and L122, polyoxylene sorbitan fatty acid esters, such as Tween 20, Tween 40, Tween 60, Tween 65, Tween 80, Tween 81, and Tween 85, and ethoxylated glycerides, such as PEG 20 almond glycerides, PEG-60 almond glycerides, PEG-20 corn glycerides, and PEG-60 corn glycerides. Generally, the carrier of the formulation of the present invention will account for about 35 wt % to about 88 wt % of the formulation. Of course, the specific type and amount of carrier included in the formulation of the present invention may vary depending on, among other factors, the anticipated subject, the therapeutic agent to be delivered, the permeation enhancer chosen, and the amount of therapeutic agent to be delivered across the mucosal membrane of the GI tract.

Where a non-ionic surfactant is used as the carrier of the formulation of the present invention, the initial viscosity of the formulation (i.e., the viscosity exhibited by the formulation as it is delivered within the GI tract) and the time required for the formulation to transition to a bioadhesive gel can be at least partially controlled through the addition of water. As water is added to a formulation having a non-ionic surfactant as the carrier, the initial viscosity of the formulation will increase. However, as water content increases, the increase in viscosity of nonionic surfactants tends to be non-linear. Often, as the water content of a nonionic surfactant exceeds a certain threshold, the viscosity of the nonionic surfactant increases rapidly as the nonionic surfactant transitions to its liquid crystal state. Thus, control of the initial viscosity of a formulation including a nonionic surfactant carrier may be limited. Nevertheless, because nonionic surfactants tend to exhibit such a threshold behavior, the time required by a nonionic surfactant carrier to transition into a bioadhesive gel may be controlled, at least in part, by including greater or lesser amounts of water in the formulation. If a relatively quick conversion is desired, a formulation including a nonionic surfactant may be provided more water, thereby placing the formulation closer to the water content threshold at which the formulation will rapidly convert to a bioadhesive gel. In contrast, if a relatively slow conversion is desired, the formulation may include less water or no water, thereby placing the formulation farther from the gelling threshold.

The formulation of the present invention may also include a viscosity reducing agent that reduces the initial viscosity of the formulation. Reducing the initial viscosity of the formulation may further facilitate spreading of the formulation of the present invention across one or more areas of the GI mucosal membrane after the formulation is delivered within the GI tract. Exemplary viscosity reducing agents that may be used in the formulation of the present invention include, but are not limited to, polyoxyethylene 5 castor oil, polyoxyethylene 9 castor oil, labratil, labrasol, capmul GMO (glyceryl mono oleate), capmul MCM (medium chain mono- and diglyceride), capmul MCM C8 (glyceryl mono caprylate), capmul MCM C10 (glyceryl mono caprate), capmul GMS-50 (glyceryl mono stearate), caplex 100 (propylene glycol didecanoate), caplex 200 (propylene glycol dicaprylate/dicaprate), caplex 800 (propylene glycol di 2-ethyl hexanoate), captex 300 (glyceryl tricapryl/caprate), captex 1000 (glyceryl tricaprate), captex 822 (glyceryl triandecanoate), captex 350 (glyceryl tricaprylate/caprate/laurate), caplex 810 (glyceryl tricaprylate/caprate/linoleate), capmul PG8 (propylene mono caprylate), propylene glycol, and propylene glycol laurate (PGL). Where a viscosity reducing agent is included in the formulation of the present invention, the viscosity reducing agent will generally account for up to about 10 wt % of the formulation. As is true of each of the other constituents of the formulation of the present invention, however, the precise amount of viscosity reducing agent included in the formulation of the present invention may be varied, as desired, to achieve a sought after therapeutic benefit.

The capability of the formulation of the present invention to transition from a relatively non-adhesive, low viscosity liquid to a viscous, bioadhesive gel in-situ is believed to impart functional advantages to the formulation of the present invention, relative to simply delivering the formulation as a bioadhesive gel. For example, it is believed that delivering the formulation as a relatively non-adhesive, low viscosity liquid enables the formulation to more easily spread across one or more areas of the GI mucosal membrane before converting to a relatively viscous, bioadhesive gel. This would allow a given volume of the formulation to present the therapeutic agent and permeation enhancer over a greater area of the GI mucosal membrane, thereby increasing the amount of therapeutic agent absorbed for a given volume of formulation. Another advantage imparted by delivering the formulation of the present invention as a relatively non-adhesive, low viscosity liquid is that doing so is believed to reduce indiscriminant adhesion of the formulation of the present invention to material contained within the GI lumen. As is easily appreciated, if the formulation was delivered as a bioadhesive substance, the formulation could indiscriminately adhere to the lumenal contents instead of the GI mucosal membrane, limiting the amount of formulation available to adhere to the GI mucosal membrane. In extreme instances, if the formulation was delivered as a bioadhesive substance, the entire volume of the formulation delivered may be encapsulated by or adhere to lumenal contents before the formulation had the opportunity to adhere to the mucosal membrane of the GI tract, and in such instances the intended benefits of the formulation would be entirely negated.

To enhance the stability of the formulation of the present invention, the formulation may include an antioxidant or a preservative. For example, an antioxidant may be used to increase the long-term stability of the therapeutic agent included in the formulation. Specific examples of antioxidants suitable for use in the formulation of the present invention include, for example, butylated hydroxytoluene (BHT), ascorbic acid, fumaric acid, malic acid, ∝-tocopherol, ascorbic acid palmitate, butylated hydroxyanisole, propyl gallate, sodium ascorbate, and sodium metabisulfate. In addition, an antioxidant or preservative included the formulation of the present invention may stabilize more than one component of the formulation. Alternatively, the formulation of the present invention may include more than one different preservative or antioxidant, each preservative or antioxidant stabilizing one or more different components of the formulation.

The present invention also includes a controlled release dosage form providing controlled delivery of the formulation of the present invention. The dosage form of the present invention contains the formulation of the present invention and is capable of delivering the formulation of the present invention at a desired release rate or release rate profile over a desired period of time. For example, a controlled release dosage form may provide a zero order, ascending, descending, or pulsatile rate of formulation release over a period of time ranging from between about 4 hours to about 24 hours. Of course, the delivery period provided by the dosage form of the present invention may be varied as desired and may fall outside the presently preferred range of about 4 hours to about 24 hours.

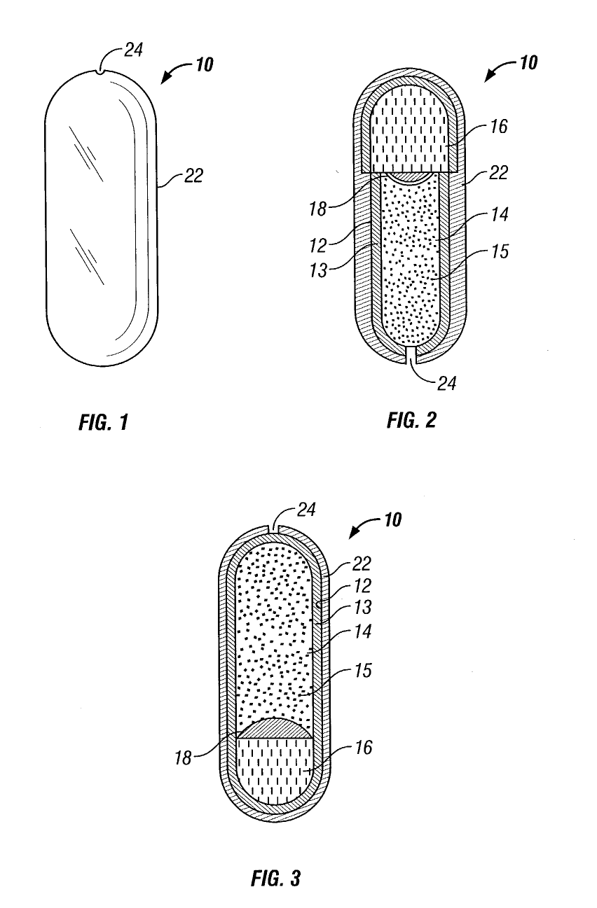

FIG. 1 through FIG. 5 illustrate various controlled release dosage forms 10 according to the present invention that utilize hard pharmaceutical capsules 12 (“hard-caps”). Where a hard-cap 12 is used to create a controlled release dosage form 10 according to the present invention, the hard-cap 12 will include a formulation 14 according to the present invention including a therapeutic agent 15, and to expel the formulation 14, the hard-cap 12 may also include an osmotic engine 16. Preferably, the osmotic engine 16 and formulation contained in a hard-cap controlled release dosage form 10 of the present invention are separated by a barrier layer 18 that is substantially fluid impermeable. In order to facilitate delivery of the formulation 14 from a hard-cap controlled release dosage form 10 of the present invention, the dosage form 10 may include an exit orifice 24, and where provided, the exit orifice 24 may only extend through the semipermeable membrane 22, or, alternatively, the exit orifice 24 may extend down through the wall 13 of the hard-cap 12. If necessary to limit or prevent undesired leakage of the formulation 14, the exit orifice 24 may be sealed using a closure 26.

Any suitable hard-cap may be used to fabricate a controlled release dosage form 10 according to the present invention. For example, U.S. Pat. No. 6,174,547, the contents of which are incorporated herein by this reference, teaches various controlled release hard-cap dosage forms, including two-piece or one-piece hard-caps that are suitable for use in the fabrication of a hard-cap controlled release dosage form according to the present invention. Moreover, U.S. Pat. No. 6,174,547 teaches various techniques useful for manufacturing two-piece and one-piece hard-caps. Materials useful for the manufacture of hard-caps useful in a dosage form according to the present invention include, for example, those materials described in U.S. Pat. No. 6,174,547, as well as other commercially available materials including gelatin, a thiolated gelatin, gelatin having a viscosity of about 15 to about 30 millipoise and a bloom strength of up to 150 grams, gelatin having a bloom value of 160 to 250, a composition comprising gelatin, glycerin, water and titanium dioxide, a composition comprising gelatin, erythrosine, iron oxide, and titanium dioxide, a composition comprising gelatin, glycerin, sorbitol, potassium sorbate, and titanium dioxide, a composition comprising gelatin, acacia, glycerin and water, and water soluble polymers that permit the transport of water there through and can be made into capsules.

The osmotic engine 16 of a hard-cap controlled release dosage 10 form of the present invention includes composition that expands as it absorbs water, thereby exerting a push-driving force against the formulation 14 and expelling the formulation 14 from the dosage form 10. The osmotic engine 16 includes a hydrophilic polymer capable of swelling or expanding upon interaction with water or aqueous biological fluids. Hydrophilic polymers are known also as osmopolymers, osmogels, and hydrogels, and will create a concentration gradient across the semipermeable membrane 22, whereby aqueous is imbibed into the dosage form 10. Hydrophilic polymers that may be used to fabricate an osmotic engine 16 useful in a controlled release dosage form 10 of the present invention include, for example, poly(alkylene oxides), such as poly(ethylene oxide), having weight average molecular weights of about 1,000,000 to about 10,000,000 and alkali carboxymethylcelluloses, such as sodium carboxymethylcellulose, having weight average molecular weights of about 10,000 to about 6,000,000. The hydrophilic polymers used in the osmotic engine 16 may be noncross-linked or cross-linked, with cross-linkages created by covalent or ionic bonds or residue crystalline regions after swelling. The osmotic engine 16 generally includes about 10 mg to about 425 mg of hydrophilic polymer. The osmotic engine 16 may also include about 1 mg to about 50 mg of a poly(cellulose), such as, for example hydroxyethylcellulose, hydroxypropylcellulose, hydroxypropylmethylcellulose, and hydroxypropylbutylcellulose. Further, the osmotic engine 16 may include about 0.5 mg to about 75 mg an osmotically effective solute, such as a salt, acid, amine, ester or carbohydrate selected from magnesium sulfate, magnesium chloride, potassium sulfate, sodium sulfate, lithium sulfate, potassium acid phosphate, mannitol, urea, inositol, magnesium succinate, tartaric acid, sodium chloride, potassium chloride, raffinose, sucrose, glucose, lactose, and sorbitol. Where included, an osmotically effective solute works to imbibe fluid through the semipermeable membrane 22 and into the dosage form 10. Optionally, the osmotic engine 16 may include 0 wt % to 3.5 wt % of a colorant, such as ferric oxide. The total weight of all components in the osmotic engine 16 is equal to 100 wt %. Of course, the osmotic engine 16 included in a controlled release dosage form according to the present invention is not limited to the exact components or the precise component weights described herein. Where included, the osmotic engine 16 is simply formulated to imbibe water into the dosage form 10 and provide a push-driving force sufficient to expel the formulation 14 as water is imbibed and the osmotic engine 16 expands.

Additional hydrophilic polymers that may be used in the osmotic engine 16 of a controlled release dosage form 10 of the present invention include: poly-(hydroxyalkyl methacrylate) having a weight average molecular weight of from 20,000 to 5,000,000; poly(vinylpyrrolidone) having a weight average molecular weight of from 10,000 to 360,000; anionic and cationic hydrogels; polyelectrolyte complexes; poly(vinyl alcohol) having a low acetate residual, cross-linked with glyoxal, formaldehyde, or glutaraldehyde and having a degree of polymerization of from 200 to 300,000; a mixture of methyl cellulose, cross-linked agar and carboxymethyl cellulose; a mixture of hydroxypropyl methycellulose and sodium carboxymethylcellulose; a mixure of hydroxypropyl ethycellulose and sodium carboxymethyl cellulose; sodium carboxymethylcellulose; postassium carboxymethylcellulose; a water insoluble, water swellable copolymer from a dispersion of finely divided copolymer of maleic anhydride with styrene, ethylene, propylene, butylenes, or isobutylene cross-linked with from 0.001 to about 0.5 miles of saturated cross-linking agent per mole of maleic anhydride per copolymer; water swellable polymers of N-vinyl lactams; polyoxyethylene-polyoxypropylene gel; polyoxybutylene-polyethylene block copolymer gel; carbo gum; polyacrylic gel; polyester gel; polyuria gel; polyether gel; polyamide gel; polycellulosic gel; polygum gel; initially dry hydrogels that imbibe and absorb water which penetrates the glass hydrogel and lowers its glass temperature; Carbopol® acidic carboxypolymer, a polymer of acrylic and cross-linked with a polyallyl sucrose, which also known as carboxypolymethylene and carboxyvinyl polymer having a weight average molecular weight of 250,000 to 4,000,000; Cyanamer® polyacrylamides; crosslinked water swellable indene-maleic anhydride polymers; Good-rite® polyacrylic acid having a weight average molecular weight of 100,000; Polyox® polyethylene oxide polymer having a weight average molecular weight of 100,000 to 7,500,000 or higher; starch graft copolymers; and Aqua-Keps® acrylate polymer polysaccharides composed of condensed glucose units such as dieters cross-linked polygluran. Further hydrophilic polymers suitable for use in a controlled release dosage form of the present invention are taught in U.S. Pat. No. 3,865,108, U.S. Pat. No. 4,002,173, U.S. Pat. No. 4,207,893, and Handbook of Common Polymers, Scott and Roff, CRC Press, Cleveland, Ohio, 1971.

Where a barrier layer 18 is provided between the osmotic engine 16 and the formulation 14, the barrier layer 18 works to minimize or prevent mixing of the formulation 14 and the osmotic engine 16 composition before and during operation of the dosage form 10. By minimizing or preventing mixing between the osmotic engine 18 and the formulation 14, the barrier layer 18 serves to reduce the amount of residual formulation 14 that remains within the dosage form 10 once the osmotic engine 18 has ceased expansion or has filled the interior of the dosage form 10. The barrier layer also serves to increase the uniformity with which the driving power of the osmotic engine 18 is transferred to the formulation 14 included in the dosage form 10. The barrier layer is made of a substantially fluid impermeable composition, such as a polymeric composition, a high density polyethylene, a wax, a rubber, a styrene butadiene, a polysilicone, a nylon, Teflon®, a polystyrene, a polytetrafluoroethylene, halogenated polymers, a blend of a microcrystalline, high acetyl cellulose, or a high molecular weight fluid impermeable polymer.

The semipermeable membrane 22 included on a controlled release dosage form 10 of the present invention is permeable to the passage of fluid, such as the aqueous biological fluid present within the GI tract of an animal or human subject, but the semipermeable membrane 22 is substantially impermeable to the passage of the formulation 14 included in the dosage form 10. The semipermeable membrane 22 is non-toxic and maintains its physical and chemical integrity during the drug delivery device of dosage form 10. Further, adjusting the thickness or chemical make-up of the semipermeable membrane 22 can control the release rate or release rate profile provided by a controlled release dosage form 10 according to the present invention. Though the semipermeable membrane 22 may be formed using any suitable material, the semipermeable membrane will generally be formed using materials that include semipermeable polymers, semipermeable homopolymers, semipermeable copolymers, and semipermeable terpolymers. Semipermeable polymers are known in the art, as exemplified by U.S. Pat. No. 4,077,407, and they can be made by procedures described in Encyclopedia of Polymer Science and Technology, Vol. 3, pages 325 to 354, 1964, published by Interscience Publishers, Inc., New York.

Cellulosic polymer materials are well suited for use in forming a semipermeable membrane 22 applied to a controlled release dosage form 10 of the present invention. Where they are used to form a semipermeable membrane 22, cellulosic polymers preferably have a degree of substitution (D.S.) on their anhydroglucose unit ranging from between greater than 0 up to 3 inclusive. As used herein, “degree of substitution” signifies the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, and semipermeable polymer forming groups.

Cellulosic polymers that may be used to form a semipermeable membrane 22 for a controlled release dosage form 10 of the present invention include, for example, cellulose esters, cellulose ethers, and cellulose ester-ethers. Typically, a cellulosic polymer used to create a semipermeable membrane 22 of a controlled release dosage form 10 of the present invention will be selected from the group including cellulose acylate, cellulose diacylate, cellulose triacetate, cellulose acetate, cellulose diacetate, cellulose triacetate, mono-, di-, and tri-cellulose alkanylates, mono-, di-, and tri-alkenylates, mono-, di, and tri-aroylates, and the like. Specific cellulosic polymer materials that may be used to form the semipermeable membrane 22 of a controlled release dosage form 10 of the present invention include, but are not limited to, the following: polymers include cellulose acetate having a D.S. of 1.8 to 2.3 and an acetyl content of 32 to 39.9%; cellulose diacetate having a D.S. of 1 to 2 and an acetyl content of 21 to 35%; and cellulose triacetate having a D.S. of 2 to 3 and an acetyl content of 34 to 44.8%; cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45% and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29.5%, a butyryl content of 17 to 53%, and a hydroxyl content of 0.5 to 4.7%; cellulose triacylates having a D.S. of 2.9 to 3 such as cellulose trivalerate, cellulose trilaurate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, and cellulose dicarpylate; and mixed cellulose esters such as cellulose acetate valerate, cellulose acetate succinate, cellulose propionate succinate, cellulose acetate octanoate, cellulose valerate palmitate, cellulose acetate heptonate.

Additional semipermeable polymers that may be used to form a semipermeable mebrane 22 included on a controlled release dosage form 10 of the present invention include the following: cellulose acetaldehyde dimethyl acetate; cellulose acetate ethylcarbamate; cellulose acetate methylcarbamate; cellulose dimethylaminoacetate; semipermeable polyamides; semipermeable polyurethanes; semipermeable sulfonated polystyrenes; cross-linked, selectively semipermeable polymers formed by the coprecipitation of a polyanion and a polycation as disclosed in U.S. Pat. Nos. 3,173,876, 3,276,586, 3,541,005, 3,541,006, and 3,546,142; semipermeable polymers disclosed by Loeb and Sourirajan in U.S. Pat. No. 3,133,132; semipermeable polystyrene derivatives; semipermeable poly(sodium styrenesulfonate); semipermeable poly(vinylbenzyltrimethyl) ammonium chloride; and semipermeable polymers exhibiting a fluid permeability of 10 to 10 (cc.mil/cm.hr.atm) expressed as per atmosphere of hydrostatic or osmotic pressure difference across a semipermeable wall. Such polymers are known to the art, as exemplified by U.S. Pat. Nos. 3,845,770, 3,916,899, and 4,160,020, and by the Handbook of Common Polymers, by Scott, J. R. and Roff, W. J., 1971, published by CRC Press, Cleveland, Ohio.

A semipermeable membrane 22 applied to a controlled release dosage form of the present invention may also include a flux regulating agent. The flux regulating agent is a compound added to assist in regulating the fluid permeability or flux through the semipermeable membrane 22. The flux regulating agent can be a flux enhancing agent or a flux reducing agent and may be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the wall when incorporated therein generally is from about 0.01% to 20% by weight or more. The flux regulating agents in one embodiment include polyhydric alcohols, polyalkylene glycols, polyalkylenediols, polyesters of alkylene glycols, and the like. Typical flux enhancers include the following: polyethylene glycol 300, 400, 600, 1500, 4000, 6000, poly(ethylene glycol-co-propylene glycol); low molecular weight gylcols such as polypropylene glycol, polybutylene glycol and polyamylene glycol; polyalkylenediols, such as poly(1,3-propanediol), poly(1,4-butanediol), poly(1,6-hexanediol); aliphatic diols, such as 1,3-butylene glycol, 1,4-pentamethylene glycol, 1,4-hexamethylene glycol; alkylene triols, such as glycerine, 1,2,3-butanetriol, 1,2,4-hexanetriol, 1,3,6-hexanetriol; and esters such as ethylene glycol dipropionate, ethylene glycol butyrate, butylene glucol dipropionate, and glycerol acetate esters. Representative flux decreasing agents include the following: phthalates substituted with an alkyl or alkoxy or with both an alkyl and alkoxy group, such as diethyl phthalate, dimethoxyethyl phthalate, dimethyl phthalate, and [di(2-ethylhexyl) phthalate]; aryl phthalates, such as triphenyl phthalate, and butyl benzyl phthalate; insoluble salts, such as calcium sulphate, barium sulphate, and calcium phosphate; insoluble oxides, such as titanium oxide; polymers in powder, granule, and like form, such as polystyrene, polymethylmethacrylate, polycarbonate, and polysulfone; esters, such as citric acid esters esterfied with long chain alkyl groups; inert and substantially water impermeable fillers; and resins compatible with cellulose based wall forming materials.

In addition, a semipermeable membrane 22 useful in a controlled release dosage form 10 of the present invention may include materials, such as a plasticizer, which impart flexibility and elongation properties to the semipermeable membrane 22. Exemplary materials that will render the semipermeable membrane 22 less brittle and impart greater tear strength to the semipermeable membrane 22, include phthalate plasticizers, such as dibenzyl phthalate, dihexyl phthalate, butyl octyl phthalate, straight chain phthalates of six to eleven carbons, di-isononyl phthalte, and di-isodecyl phthalate. Suitable plasticizers further include, for example, nonphthalates, such as triacetin, dioctyl azelate, epoxidized tallate, tri-isoctyl trimellitate, tri-isononyl trimellitate, sucrose acetate isobutyrate, and epoxidized soybean oil. Where incorporated in a semipermeable membrane 22, a plasticizer will generally account for about 0.01 wt % to about 20 wt %, or higher, of the membrane formulation.

The expression “exit orifice” as used herein comprises means and methods suitable for releasing the formulation 14 contained within a controlled release dosage form 10 of the present invention. An exit orifice 24 included in a controlled release dosage form 10 according to the present invention may include a passageway, aperture, hole, bore, pore, and the like through the semipermeable membrane 22, or through the semipermeable membrane 22 and the wall 13 of the capsule 12 used to form the controlled release dosage form 10. Alternatively, the exit orifice 24 may include, for example, a porous element, porous overlay, porous insert, hollow fiber, capillary tube, microporous insert, or microporous overlay. The exit orifice 24 can be formed by mechanical drilling or laser drilling, by eroding an erodible element, such as a gelatin plug or a pressed glucose plug, or by crimping the walls to yield the exit orifice 24 when the dosage form is in the environment of use. In an embodiment, the exit orifice 24 in wall 13 is formed in the environment of use in response to the hydrostatic pressure generated within the controlled release dosage form 10. If desired or necessary, the controlled release dosage form 10 can be manufactured with two or more exit orifices (not shown) for delivering formulation 14 during use. A detailed description of orifices and exemplary maximum and minimum dimensions of exit orifices used in controlled release dosage form are disclosed in U.S. Pat. Nos. 3,845,770, 3,916,899, and 4,200,098, the contents of which are herein incorporated by this reference.

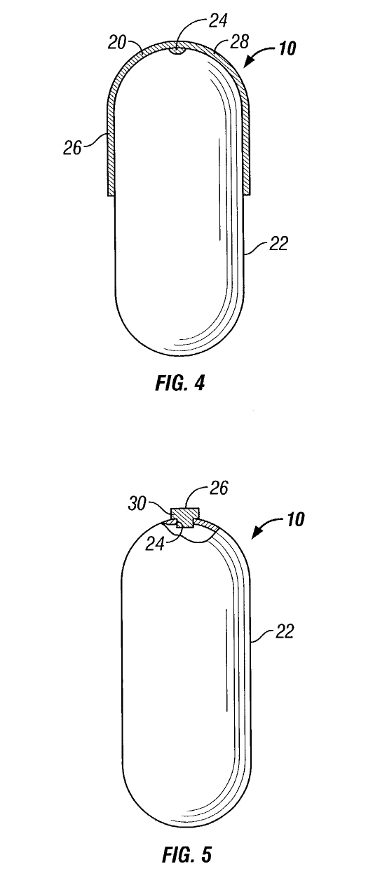

If included in a controlled release dosage form 10 of the present invention, a closure 26 sealing the exit orifice 24 may be provided by any one of several means. For instance, as illustrated in FIG. 4, the closure 26 may simply include a layer 28 of material that covers the exit orifice 24 and is arranged over a portion of the lead end 20 of the dosage form. Alternatively, as shown in FIG. 5, closure 26 may include a stopper 30, such as a bung, cork, or impermeable plug, formed or positioned within the exit orifice 24. Regardless of its specific form, the closure 26 comprises a material impermeable to the passage of fluid, such as high density fluid impermeable polyolefin aluminized polyethylene, rubber, silicon, nylon, synthetic fluorine Teflon®, chlorinated hydrocarbon polyolefins, and fluorinated vinyl polymers. Further, where included, the closure 26 may formed in any suitable shape using any suitable manufacturing technique.

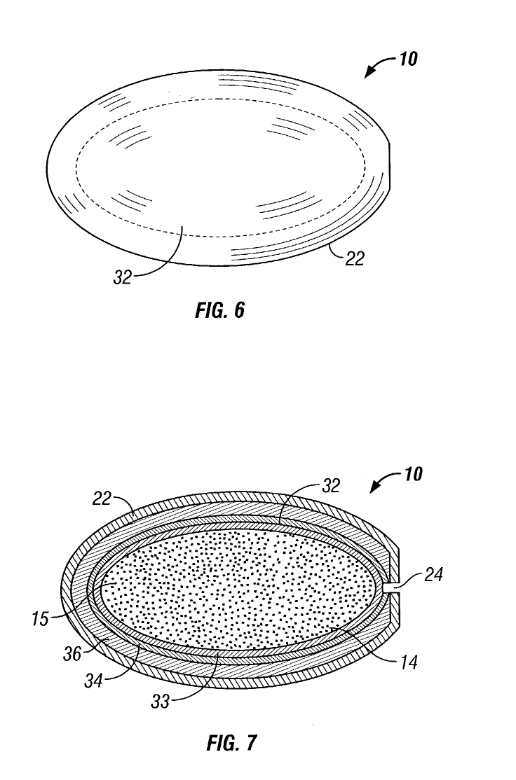

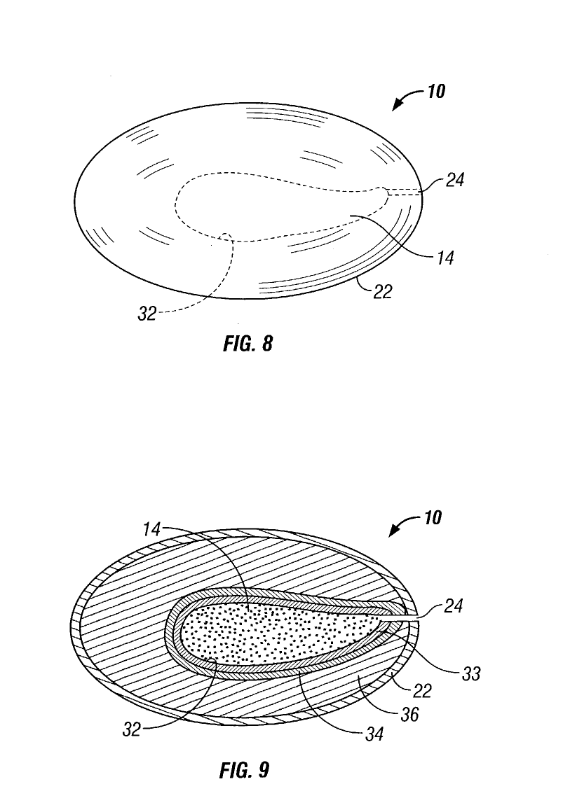

The controlled release dosage form of the present invention may also be formed using a soft gelatin capsule (soft-cap), shown in FIG. 6-FIG. 19. Where a soft-cap is used to form the controlled release dosage form 10 of the present invention, the dosage form 10 includes a soft-cap 32 containing a formulation 14 of the present invention including a therapeutic agent 15. A barrier layer 34 is formed around the soft-cap 32, and an osmotic layer 36 is formed around the barrier layer 34. Like the hard-cap controlled release dosage form already described, a soft-cap controlled release dosage form 10 according to the present invention is also provided with a semipermeable membrane 22, the semipermeable membrane 22 being formed over the osmotic layer 36. An exit orifice 24 is preferably formed through the semipermeable membrane 22, the osmotic layer 36, and the barrier layer 34 to facilitate delivery of the formulation 14 from the soft-cap controlled release dosage form 10.

The soft-cap 32 used to create a controlled release dosage form 10 of the present invention may be a conventional gelatin capsule, and may be formed in two sections or as a single unit capsule in its final manufacture. Preferably, due to the presence of the barrier layer 34, the wall 33 of the soft-cap 32 retains its integrity and gel-like characteristics, except where the wall 33 dissolves in the area exposed at the exit orifice 24. Generally maintaining the integrity of the wall 33 of the soft-cap 32 facilitates well-controlled delivery of the formulation 14. However, some dissolution of portions of the soft-cap 32 extending from the exit orifice 24 during delivery of the formulation 14 may be accommodated without significant impact on the release rate or release rate profile of the formulation 14.

Any suitable soft-cap may be used to form a controlled release dosage form according to the present invention. The soft-cap 32 may be manufactured in accordance with conventional methods as a single body unit comprising a standard capsule shape. Such a single-body soft-cap typically may be provided in sizes from 3 to 22 minims (1 minimim being equal to 0.0616 ml) and in shapes of oval, oblong, or others. The soft cap 32 may also be manufactured in accordance with conventional methods as a two-piece hard gelatin capsule that softens during operation, such as by hydration. Such capsules are typically manufactured in standard shapes and various standard sizes, conventionally designated as (000), (00), (0), (1), (2), (3), (4), and (5), with largest number corresponding to the smallest capsule size. However, whether the soft-cap 32 is manufactured using soft gelatin capsule or hard gelatin capsule that softens during operation, the soft-cap 32 may be formed in non-conventional shapes and sizes if required or desired for a particular application.

At least during operation, the wall 33 of the soft-cap 32 should be soft and deformable to achieve a desired release rate or release rate profile. The wall 33 of a soft-cap 32 used to create a controlled release dosage form 10 according to the present invention will typically have a thickness that is greater than the thickness of the wall 13 of a hard-cap 12 used to create a hard-cap controlled release dosage form 10. For example, soft-caps may have a wall thickness on the order of 10-40 mils, with about 20 mils being typical, whereas hard-caps may have a wall thickness on the order of 2-6 mils, with about 4 mils being typical. U.S. Pat. No 5,324,280 describes the manufacture of various soft-caps useful for the creation of controlled release dosage form according to the present invention, and the contents of U.S. Pat. No. 5,324,280 are herein incorporated by this reference.

The barrier layer 34 formed around the soft-cap 32 is deformable under the pressure exerted by the osmotic layer 36 and is preferably impermeable (or less permeable) to fluids and materials that may be present in the osmotic layer 36 and in the environment of use during delivery of the formulation 14 contained within the soft-cap 32. The barrier layer 34 is also preferably impermeable (or less permeable) to the formulation 14 of the present invention. However, a certain degree of permeability of the barrier layer 34 may be permitted if the release rate or release rate profile of the formulation 14 is not detrimentally affected. As it is deformable under forces applied by osmotic layer 36, the barrier layer 34 permits compression of the soft-cap 32 as the osmotic layer 36 expands. This compression, in turn, forces the formulation 14 from the exit orifice 24. Preferably, the barrier layer 34 is deformable to such an extent that the barrier layer 34 creates a seal between the osmotic layer 36 and the semipermeable layer 22 in the area where the exit orifice 24 is formed. In that manner, barrier layer 34 will deform or flow to a limited extent to seal the initially exposed areas of the osmotic layer 36 and the semipermeable membrane 22 when the exit orifice 24 is being formed.

Suitable materials for forming the barrier layer 34 include, for example, polyethylene, polystyrene, ethylene-vinyl acetate copolymers, polycaprolactone and Hytrel® polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease® as supplied by Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit® supplied by RöhmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials for the formation of the barrier layer 34 include, for example, cellulose acetate, copolymers of acrylic acid and methacrylic acid esters, copolymers of methylmethacrylate and ethylacrylate, and latex of acrylate esters. Preferred copolymers include the following: poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30D; poly (methacrylic acid, methyl methacrylate) 1:1, 135,000, sold under the trademark EUDRAGIT L; poly (methacrylic acid, ethyl acrylate) 1:1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; and poly (ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

Where desired, a plasticizer may be compounded with the material used to fabricate the soft-cap 32 or the barrier layer 34. Inclusion of a plasticizer increases the flow prospects of the material and enhances the workability of the material during manufacture of the soft cap 32 or the barrier layer 34. For example, glycerin can be used for plasticizing gelatin, pectin, casein or polyvinyl alcohol. Other plasticizers that can be used for the present purpose include, for example, triethyl citrate, diethyl phthalate, diethyl sebacate, polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. Where included, the amount of plasticizer in a formulation used to create a soft-cap 32 will generally range from about 0.05 wt % to about 30 wt %, while the amount of plasticizer in a formulation used to create a barrier layer 34 may be as high as about 10 wt % to about 50 wt %.

The osmotic layer 36 included in a soft-cap controlled release dosage form 10 according to the present invention includes a hydro-activated composition that expands in the presence of water, such as that present in gastric fluids. The osmotic layer 36 may be prepared using those materials suitable for producing the osmotic engine of the hard-cap controlled release dosage form previously described. As the osmotic layer 36 imbibes and/or absorbs external fluid, it expands and applies a pressure against the barrier layer 34 and the wall 33 of the gel-cap 32, thereby forcing the formulation 14 through the exit orifice 24.

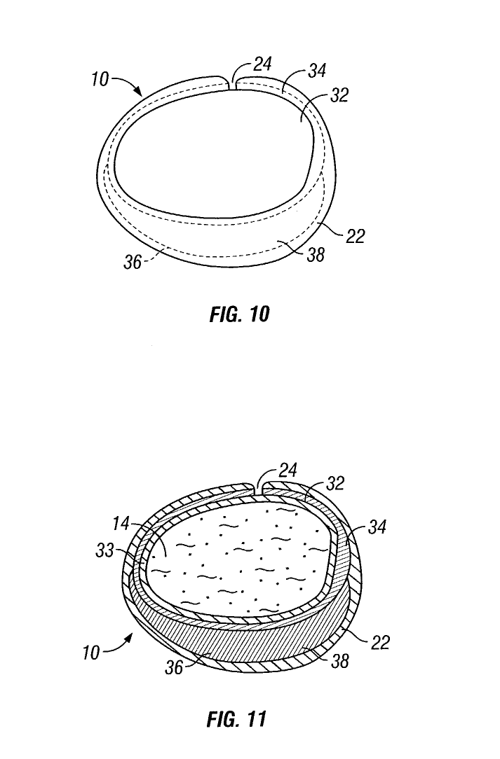

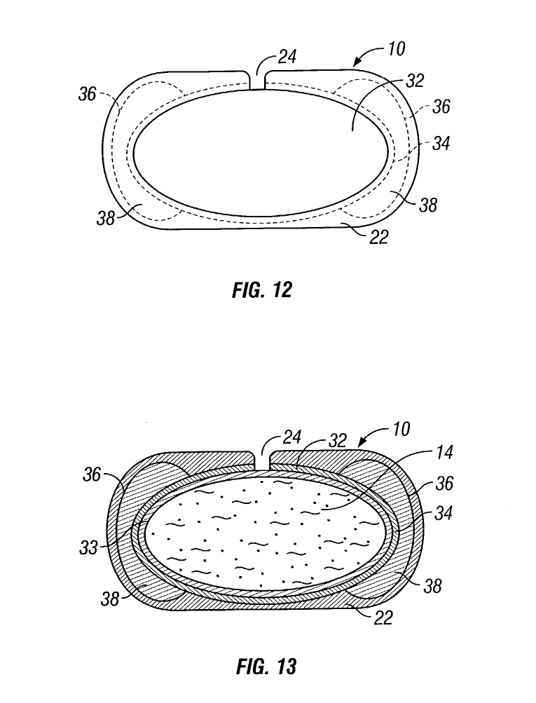

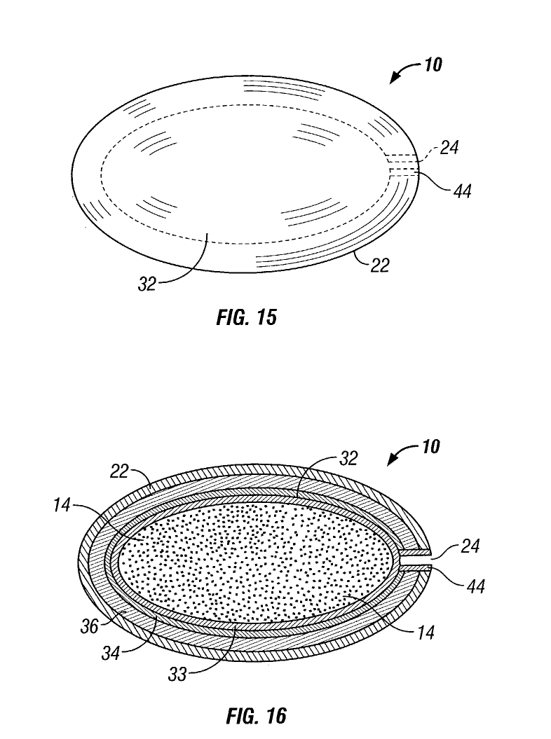

As shown in FIG. 6, FIG. 10-FIG. 13, and FIG. 15-FIG. 16, the osmotic layer 36 included in a soft-cap controlled release dosage form 10 of the present invention may be configured as desired to achieve a desired release rate or release rate profiles, as well as a desired delivery efficiency. For example, the osmotic layer 36 may be an unsymmetrical hydro-activated layer (shown in FIG. 10 and FIG. 11), having a thicker portion remote from the exit orifice 24. The presence of the unsymmetrical hydro-activated layer functions to assure that the maximum dose of formulation 14 is delivered from the dosage form 10, as the thicker section of the osmotic layer 36 swells and moves towards the exit orifice 24. As is easily appreciated by reference to the figures, the osmotic layer 36 may be formed in one or more discrete sections 38 that do not entirely encompass the barrier layer 34 formed around the soft cap 32 (shown in FIG. 10-FIG. 13). As can be seen from FIG. 10 and FIG. 11, the osmotic layer 36 may be a single element 40 that is formed to fit the shape of the soft-cap 32 at the area of contact. Alternatively, the osmotic layer 36 may include two or more discrete sections 38 formed to fit the shape of the soft-cap 32 in the areas of contact (shown in FIG. 12 and FIG. 13).

The osmotic layer 36 may be fabricated using know materials and know fabrication techniques. For example, the osmotic layer maybe fabricated conveniently by tableting to form an osmotic layer 36 of a desired shape and size. For example, the osmotic layer 36 may be tableted in the form a of a concave surface that is complementary to the external surface of the barrier layer 34 formed on the soft-cap 32. Appropriate tooling such as a convex punch in a conventional tableting press can provide the necessary complementary shape for the osmotic layer. Where it is formed by tableting, the osmotic layer 36 is granulated and compressed, rather than formed as a coating. Methods of forming an osmotic layer by tableting are described, for example, in U.S. Pat. Nos. 4,915,949, 5,126,142, 5,660,861, 5,633,011, 5,190,765, 5,252,338, 5,620,705, 4,931,285, 5,006,346, 5,024,842, and 5,160,743, the contents of which are incorporated herein by this reference.

The semipermeable membrane 22 formed around the osmotic layer 36 is non-toxic and maintains its physical and chemical integrity during operation of the soft-cap controlled release dosage form 10. The semipermeable membrane 22 is created using a comprising a composition that does not adversely affect the subject or the other components of the soft-cap controlled release dosage form 10. The semipermeable membrane 22 is permeable to the passage of fluid such as water and biological fluids, but it is substantially impermeable to the passage of the formulation 14 contained within the soft-cap 32 and of the materials forming the osmotic layer 36. For ease of manufacture, it is preferred that the whole of the layer formed around the osmotic layer 36 be a semipermeable membrane 22. The semipermeable compositions used for forming the semipermeable membrane 22 are essentially non-erodible, and they are insoluble in biological fluids during the operational lifetime of the osmotic system. Those materials already set forth as suitable for forming the semipermeable membrane 22 of the previously described hard-cap controlled release dosage form 10 are also suitable for forming the semipermeable membrane 22 of a soft-cap controlled release dosage form 10. The release rate or release rate profile of a soft-cap controlled release dosage form 10 can be controlled by adjusting the thickness or chemical make-up of the semipermeable membrane 22.

The barrier layer 34, osmotic layer 36, and semipermeable layer 22 may be applied to the exterior surface of the soft-cap 32 by conventional coating procedures. For example, conventional molding, forming, spraying, or dipping processes may be used to coat the soft-cap with each layer forming composition. An air suspension procedure that may be used to coat one or more layers on a controlled release dosage form of the present invention is described in U.S. Pat. No. 2,799,241; J. Am. Pharm. Assoc., Vol. 48, pp. 451-59, 1979; and ibid, Vol. 49, pp. 82-84,1960. Other standard manufacturing procedures are described in Modern Plastic Encyclopedia, Vol. 46, pp. 62-70, 1969; and in Pharmaceutical Sciences, by Remington, 18th Ed., Chapter 90, 1990, published by Mack Publishing Co., Easton, Pa.

Exemplary solvents suitable for manufacturing the various layers of the controlled release soft-cap dosage form 10 of the present invention include inert inorganic and organic solvents that do not adversely harm the materials, the soft-cap, or the final laminated composite structure. The solvents broadly include, for example, members selected from the group consisting of aqueous solvents, alcohols, ketones, esters, ethers, aliphatic hydrocarbons, halogenated solvents, cycloaliphatic, aromatics, heterocyclic solvents and mixtures thereof. Specific solvents that may be used to manufacture the various layers of the soft-cap controlled release dosage form 10 of the present invention include, for example, acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts, such as sodium and acetone and water, acetone and methanol, acetone and ethyl alcohol, methylene dichloride and methanol, and ethylene dichloride and methanol.

In a preferred embodiment, the exit orifice 24 of a soft-cap controlled release dosage form 10 of the present invention will extend only through the semipermeable layer 22, the osmotic layer 36, and the barrier layer 34 to the wall 33 of the soft cap 32. However, the exit orifice 24 may extend partially into the wall 33 of soft cap 32, as long as the exit orifice 24 does not completely traverse the wall 33. When exposed to the environment of use, the fluids in the environment of use may dissolve the wall 33 of the soft-cap 32 where the soft-cap 32 is exposed at the exit orifice 24, or the pressure exerted on the soft-cap 32 and the barrier layer 34 by the osmotic layer 36 may cause the wall 33 of the gel-cap 32 to rupture where it is exposed to the exit orifice 24. In either case, the interior of the gel-cap 32 will be placed in fluid communication with the environment of use, and the formulation 14 will be dispensed through exit orifice 24 as the barrier layer 34 and the soft-cap 32 are compressed.

The exit orifice 24 formed in the soft-cap controlled release dosage form 10 can be formed by mechanical drilling, laser drilling, eroding an erodible element, extracting, dissolving, bursting, or leaching a passageway former from the composite wall. The passageway can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material to the desired depth to form the exit orifice 24.

It is presently preferred that a soft-cap controlled-release dosage form 10 of the present invention include mechanism for sealing any portions of the osmotic layer 36 exposed at the exit orifice 24. Such a sealing mechanism prevents the osmotic layer 36 from leaching out of the system during delivery of formulation 14. In one embodiment, the exit orifice 24 is drilled and the exposed portion of the osmotic layer 36 is sealed by barrier layer 34, which, because of its rubbery, elastic-like characteristics, flows outwardly about the inner surface of exit orifice 24 during and/or after the formation of the exit orifice 24. In that manner, the barrier layer 34 effectively seals the area between the osmotic layer 34 and semipermeable layer 22. This can be seen most clearly in FIG. 9. In order to flow and seal, the barrier layer 34 should have a flowable, rubbery-like consistency at the temperature at which the system operation takes place. Materials, such as copolymers of ethyl acrylate and methyl methacrylate, especially Eudragit NE 30D supplied by RohmPharma, Darmstaat, Germany, are preferred. A soft-cap controlled release dosage form 10 having such a sealing mechanisms may be prepared by sequentially coating the soft-cap 32 with a barrier layer 34, an osmotic layer 36, and semipermeable layer 22 and then drilling the exit orifice 24 to complete the dosage form 10.

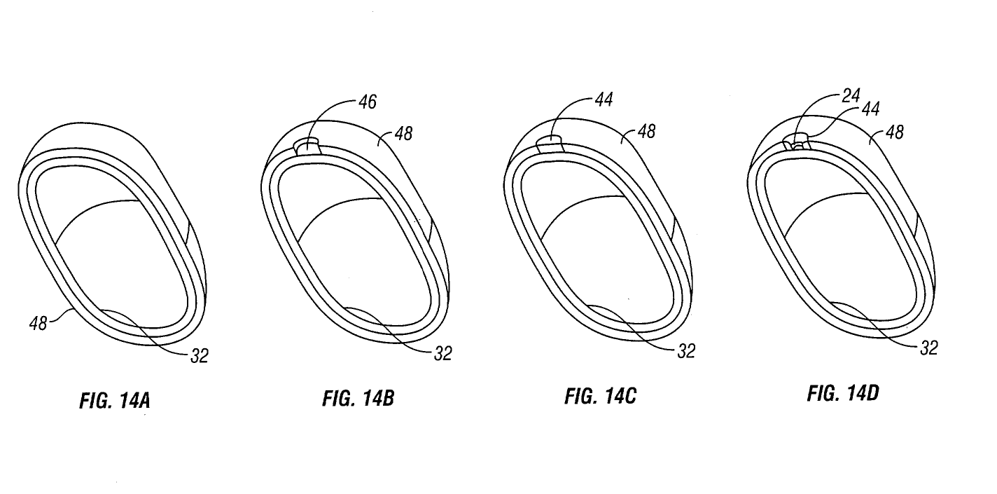

Alternatively a plug 44 may be used to form the desired sealing mechanism for the exposed portions of the osmotic layer 36. As is shown in FIG. 14A through FIG. 14D, a plug 44 may be formed by providing a hole 46 in the semipermeable membrane and the barrier layer (shown as a single composite membrane 48). The plug 44 is then formed by filling the hole 46 with, for example, a liquid polymer that can be cured by heat, radiation or the like (shown in FIG. 14C). Suitable polymers include polycarbonate bonding adhesives and the like, such as, for example, Loctite® 3201, Loctite® 3211, Loctite® 3321 and Loctite® 3301, sold by the Loctite Corporation, Hartford, Conn. The exit orifice 24 is drilled into plug to expose a portion of the soft-cap 32. A completed dosage form having a plug-type seal is illustrated in an overall view of FIG. 15 and in cross-section in FIG. 16.

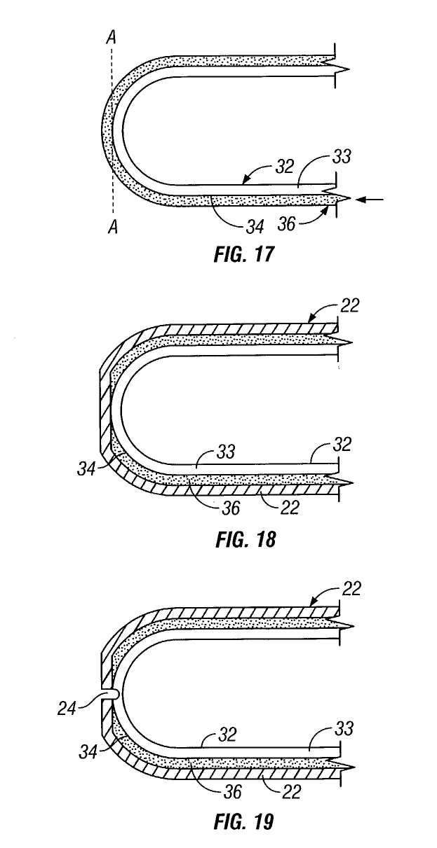

Still another manner of preparing a dosage form having a seal formed on the inner surface of the exit orifice is described with reference to FIG. 17-FIG. 19. In FIG. 17, a soft-cap 32 (only partially shown) has been coated with the barrier layer 34 and an osmotic layer 36. Prior to coating the semipermeable membrane 22, a section of the osmotic layer 36 extending down to, but not through, the barrier layer 34 is removed along line A-A. Then a semipermeable membrane 22 is coated onto the dosage form 10 to yield a precursor of the dosage form such as illustrated in FIG. 18. As can be seen from FIG. 18, the portion of gel-cap 32 where the exit orifice 24 is to be formed is covered by the semipermeable membrane 22 and the barrier layer 34, but not the osmotic layer 36. Consequently, when an exit orifice 24 is formed in that portion of the dosage form 10, as can be seen most clearly in FIG. 19, the barrier layer 34 forms a seal at the juncture of the semipermeable membrane 22 and expandable layer 20 such that fluids may pass to osmotic layer 36 only through the semipermeable membrane 22. Accordingly, osmotic layer 36 is not leached out of the dosage form 10 during operation. The sealing aspect of the soft-cap controlled release dosage form 10 of the present invention allows the rate of flow of fluids to the osmotic layer 36 to be carefully controlled by controlling the fluid flow characteristics of the semipermeable membrane 22.

The various layers forming the barrier layer, expandable layer (when not a tableted composition) and semipermeable layer may be applied by conventional coating methods such as described in U.S. Pat. No. 5,324,280, previously incorporated herein by reference. While the barrier layer, expandable layer and semipermeable layer forming the multilayer wall superposed on the soft-cap have been illustrated and described for convenience as single layers, each of those layers may be composites of several layers. For example, for particular applications it may be desirable to coat the soft-cap with a first layer of material that facilitates coating of a second layer having having the permeability characteristics of the barrier layer. In that instance, the first and second layers comprise the barrier layer as used herein. Similar considerations would apply to the semipermeable layer and the expandable layer.

In the embodiment shown in FIG. 10 and FIG. 11, the barrier layer 34 is first coated onto the gelatin capsule 12 and then the tableted, osmotic layer 36 is attached to the barrier-coated soft-cap with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form is then coated with a semipermeable membrane. The exit orifice 24 is formed in the side or end of the soft-cap 32 opposite the osmotic layer 36. As the osmotic layer 36 imbibes fluid, it will swell. Since it is constrained by the semipermeable membrane 22, the osmotic layer 36 compresses the soft-cap 32 as the osmotic layer 36 expands, thereby expressing the formulation 14 from the interior of the soft-cap 32 into the environment of use.

As mentioned, the soft-cap controlled release dosage form 10 of the present invention may include an osmotic layer formed of a plurality of discrete sections. Any desired number of discrete sections may be used, but typically the number of discrete sections will range from 2 to 6. For example, two sections 38 may be fitted over the ends of the barrier-coated soft-cap 32 as illustrated in FIG. 12 and FIG. 13. FIG. 12 is a schematic of a soft-cap controlled release dosage form 10 with the various components of the dosage form indicated by dashed lines and the soft-cap 32 indicated by a solid line. FIG. 13 is a cross-sectional view of a completed soft-cap controlled release dosage form 10 having two, discrete expandable sections 38. Each expandable section 38 is conveniently formed by tableting from granules and is adhesively attached to the barrier-coated soft-cap 32, preferably on the ends of the soft-cap 32. Then a semipermeable layer 22 is coated on the intermediate structure and an exit orifice 24 is formed in a side of the dosage form between the expandable sections 38. As the expandable sections 38 expand, the formulation 14 will be expressed from the interior of the soft-cap 32 in a controlled manner to provide controlled-release delivery of the formulation 14.

The hard-cap and soft-cap controlled release dosage forms prepared in accordance with the present invention may be constructed as desired to provide controlled release of the formulation of the present invention at a desired release rate or release rate profile over a desired period of time. Preferably, the dosage forms of the present invention are designed to provide controlled release of the formulation of the present invention over a prolonged period of time. As used herein, the phrase “prolonged period of time” indicates a period of time of two or more hours. Typically for human and veterinary pharmaceutical applications, a desired prolonged period of time may be from 2 hours to 24 hours, more often 4 hours to 18 hours or 6 hours to 12 hours. For many applications it may be preferable to provide dosage forms that only need to be administered once-a-day. Additional controlled release dosage forms that may be used to deliver the formulation of the present invention are described in U.S. Pat. Nos. 4,627,850 and 5,413,572, the contents of which are incorporated herein by this reference.

If desired, the dosage form of the present invention may be provided with an enteric coating. Enteric coatings will remain intact in the stomach, but will start dissolving once they arrive at the small intestine, allowing a dosage form to release its contents at one or more sites downstream in the intestine (e.g., the ileum and the colon). Enteric coatings are known in the art and are discussed at, for example, Remington's Pharmaceutical Sciences, (1965), 13^{th} ed., pages 604-605, Mack Publishing Co., Easton, Pa.; Polymers for Controlled Drug Delivery, Chapter 3, CRC Press, 1991; Eudragit® Coatings Rohm Pharma, (1985); and U.S. Pat. No. 4,627,851. Where a the dosage form of the present invention is provided with an enteric coating, the thickness and chemical constituents of the enteric coating may be selected to target release of the formulation of the present invention within a specific region of the lower GI tract. However, because the therapeutic agent included in the formulation of the present invention is well absorbed in the upper GI tract, the controlled release dosage form of the present invention may be designed to begin release of the formulation of the present invention in the upper GI tract, and the dosage form of the present invention need not include an enteric-coating.

The controlled release dosage form of the present invention provides functional advantages not achievable by oral dosage forms providing a dose-dumping or bolus release of the therapeutic agents included in the formulation of the present invention. Because the formulation of the present invention enhances the absorption of the therapeutic agent included in the formulation in the lower GI tract, the dosage form of the present invention can be designed to provide controlled release of the formulation of the present invention for a period of time that is longer than the anticipated transit time of the dosage form through the upper GI tract. Providing such enhanced control over the release of the formulation of the present invention facilitates increased control of the plasma concentration of the therapeutic agent included formulation of the present invention, which eases the task of achieving and maintaining therapeutic levels of the therapeutic agent within the subject. In addition, greater control of the plasma concentration of the therapeutic agent may also ease or eliminate side affects resulting from the administration of the therapeutic agent. Therefore, the dosage form and formulation of the present invention facilitate the controlled release of therapeutic agents that may not otherwise benefit from controlled release from an oral dosage form due to their poor absorption in the lower GI tract.

### EXAMPLE 1

To better appreciate the behavior of the carrier included in the formulation of the present invention, the rheological properties of an exemplary carrier, Cremophor EL (ethoxylated castor oil), were characterized. To characterize the Theological behavior of Cremophor EL, the carrier was mixed homogeneously with water in various ratios, and the Cremophor EL/water blends were measured by a Haak 100 RheoStress Rheometer for η (dynamic viscosity), G′ (storage modulus), G″ (loss modulus), and δ (G″/G′).

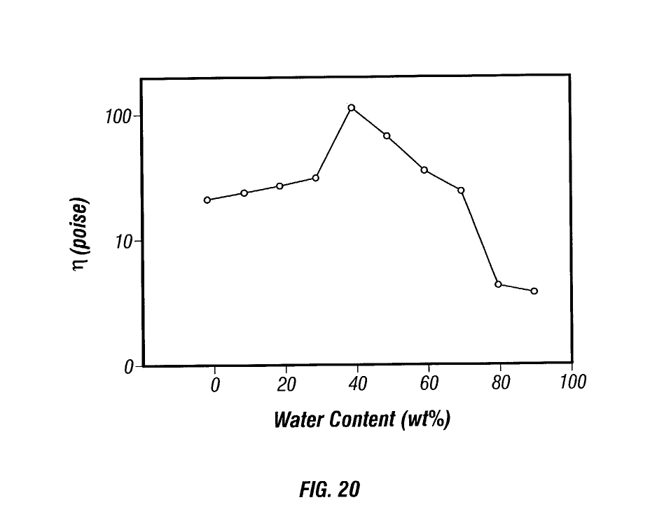

FIG. 20 shows the dynamic viscosity of various Cremophor EL/water blends as a function of water content. As can be appreciated by reference to FIG. 20, as the water content rose beyond about 30%, the viscosity of the blends increased dramatically, peaking at about 40% water content. However, as the water content continued to increase beyond about 40%, the viscosity of the blends began to decrease. As the water content approached 80%, the viscosity of the blends decreased well below the viscosity of Cremophor EL that is substantially free of water.

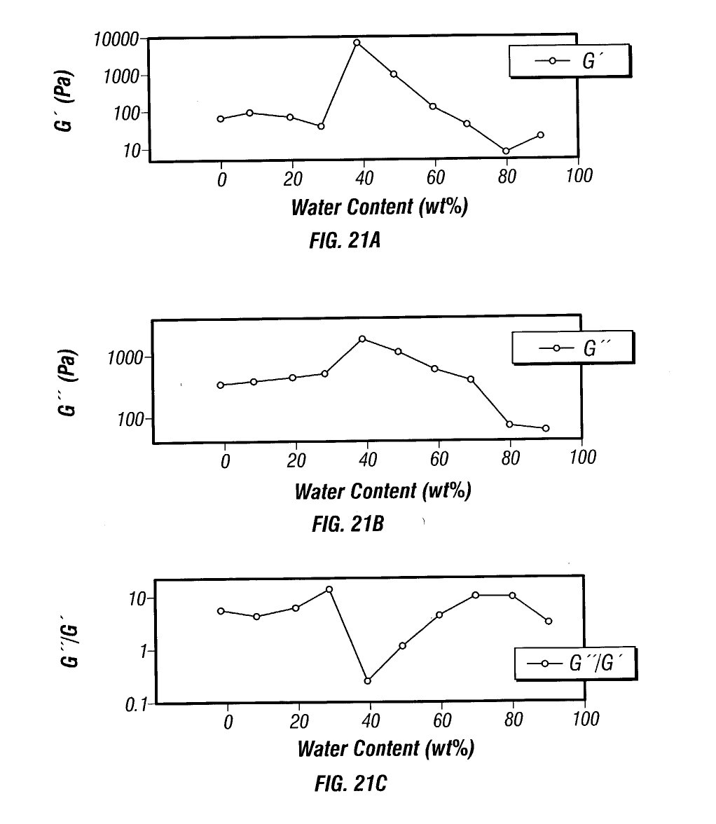

FIG. 21 shows the G′ (storage modulus), G″ (loss modulus), and δ (G″/G′) of Cremophor EL/water blends as a function of water content. As the water content of the blends rose, the Theological properties of the blends changed, significantly. In particular, as water content rose from about 30% to about 40%, the value of G″/G′ transitioned from greater than one (G″/G′>1) to less than one (G″/G′<1), indicating that Cremophor EL transitions from a liquid-type substance to a rubber-type substance as it absorbs water. However, as the water content of the blends rose beyond 40%, the value of G″/G′ transitioned back from less than one (G″/G′<1) to greater than one (G″/G′>1), which indicates that, as the water content of Cremophor EL increases beyond about 40%, the material transitions back from a rubber-like substance to a liquid-type substance.

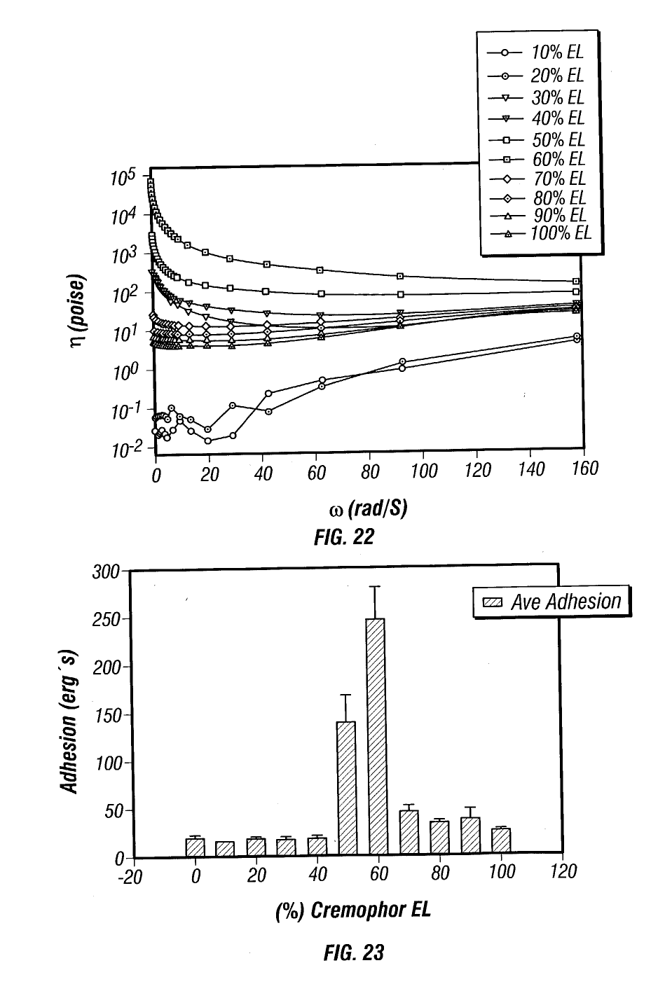

The dynamic viscosity of various Cremophor EL/water blends were measured at shear rates ranging from 0.0628 rad/s to 628 rad/s. As shown in FIG. 22, shear rate had an inverse effect on the dynamic viscosity of samples containing 30% to 60% Cremophor EL. It was demonstrated that dynamic viscosity decreased as shear rate increased, which is characteristic of the pseudoelastic behavior of non-Newtonian fluid. Other compositions of Cremophor EL/water (low viscosity) showed dilatant property (i.e., dynamic viscosity increased as shear rate increased).

In order to assess the bioadhesive properties of the Cremophor EL as a function of water content, the adhesion of various Cremophor EL/water blends to a mucin surface was determined using a texture profile analyzer (TPA) from Texture Technologies Corp. A 500 mg mucin tablet with a flat circular surface area of 0.096 in2 was compressed with a 0.5 ton force. The mucin tablet was firmly attached to the lower end of the TPA probe using double-sided adhesive tape. Samples of Cremophor EL/water blends of various ratios were prepared in small bottles that were affixed onto the TPA platform. The mucin tablet was moistened in AGF for 60 seconds prior to the measurements. During measurement, the TPA probe with attached mucin tablet was lowered onto the surface of each sample at a constant speed of 1 mm/sec. To ensure the intimate contact between the mucin tablet and the sample, the tabled stayed for 60 seconds before the probe was moved upward. The force required to detach the mucin tabled from the surface of the samples was recorded as a function of time. Adhesion energy (E) was calculated from the AUC of the curve (E=AUC×S). FIG. 23 presents the results of the measurements. The blend of Cremophor EL/water in the ratio of 60/40 was most adhesive to the surface of the mucin tablet. These results show good correlation between adhesion and viscosity, with the more viscous formulations tending to be the most adhesive as well.

### EXAMPLE 2

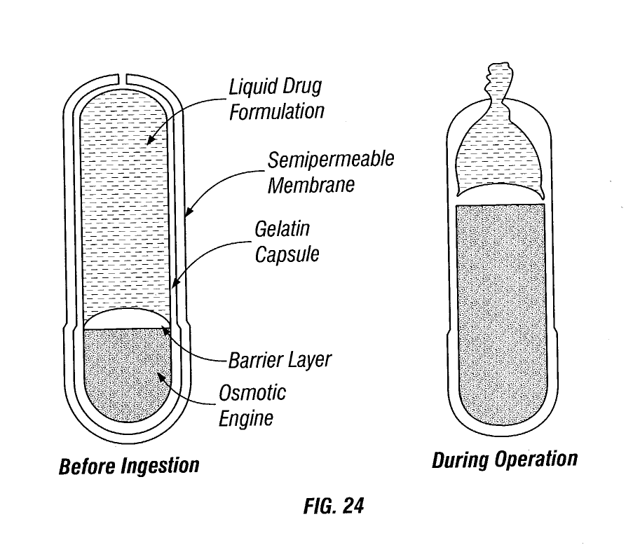

A dosage form according to the present invention including an exemplary formulation according to the present invention was manufactured. A schematic representation of the dosage form is provided in FIG. 24. As can be seen in FIG. 24, the dosage form included a gelatin capsule containing an osmotic engine, a barrier layer, and a formulation according to the present invention. A semipermeable membrane was provided on the exterior of the gelatin capsule. During operation, the osmotic engine absorbed water from the environment and expanded such that the formulation was expelled through an exit provided in the capsule at a desired, controlled rate.

To manufacture the dosage form, the osmotic engine was granulated with a Glatt fluid bed granulator (FBG). NaCl was first sized/screened using a Quadro mill with a 21 mesh screen and the speed set on maximum. Once the NaCl had been sized/screened, the following dry ingredients were added into the granulator bowl: 58.75% NaCMC, 30% sized/screen NaCl, 5.0% HPMC E-5, and 1.0% red ferric oxide. The ingredients were blended in the bowl. In a separate container, the granulating solution was prepared by dissolving 5.0% HPC EF in purified water. The granulating solution was spayed onto the fluidized powders until all of the solution was applied and the powders were granular. 0.25% MG stearate was blended with the granules.

After, the osmotic engine granules had been prepared, the osmotic engine granules and barrier layer granules, which include 90 wt % Microfine wax and 10 wt % HPMC E5, were compressed into a bilayer tablet using an appropriate tableting press, such as a Carver Press or a Manesty tableting press. To create the bilayer tablet, 250 mg of the osmotic engine and 30 mg of the barrier layer were added to a 0.277 inch punch having a modified ball lower punch and a modified ball upper punch. The ingredients were then tamped and compressed into a contacting core under a force of about 1 metric ton.

Once the osmotic engine and the barrier layer were formed into a bilayer tablet, a formulation according to the present invention was mixed and the gelatin capsule of the dosage form was filled. The formulation included, in weight percent, 50% acyclovir, 14% lauric acid, and 36% Cremophor EL. The formulation was mixed homogeneously using a suitable means, such as a homogenizer or mechanical agitator. The gelatin capsule (clear size 0) of the dosage form was separated into two segments (body and cap), and the body was filled with 600 mg of the mixed formulation. The bilayer tablet was then placed on top of the mixed formulation, with the barrier layer side of the bilayer tablet in contact with the mixed formulation. The filled capsule body was then closed with the capsule cap.

The filled gelatin capsule was provided with a semipermeable membrane. The composition used to create the semipermeable membrane included 80% cellulose acetate 398-10 and 20% Pluronic F-68, the composition being dissolved in acetone until a coating solution having a solid content of 4% was achieved. The coating solution was sprayed onto the pre-filled gelatin capsules in a 12″ Freud Hi-Coater, until a 43 mg semipermeable membrane was achieved.

After membrane coating, the dosage form was dried in a Hotpack oven at 30° C. overnight. To facilitate delivery of the formulation contained within the dosage form, an orifice was cut at the drug layer side using a 100 mil mechanical cutter.

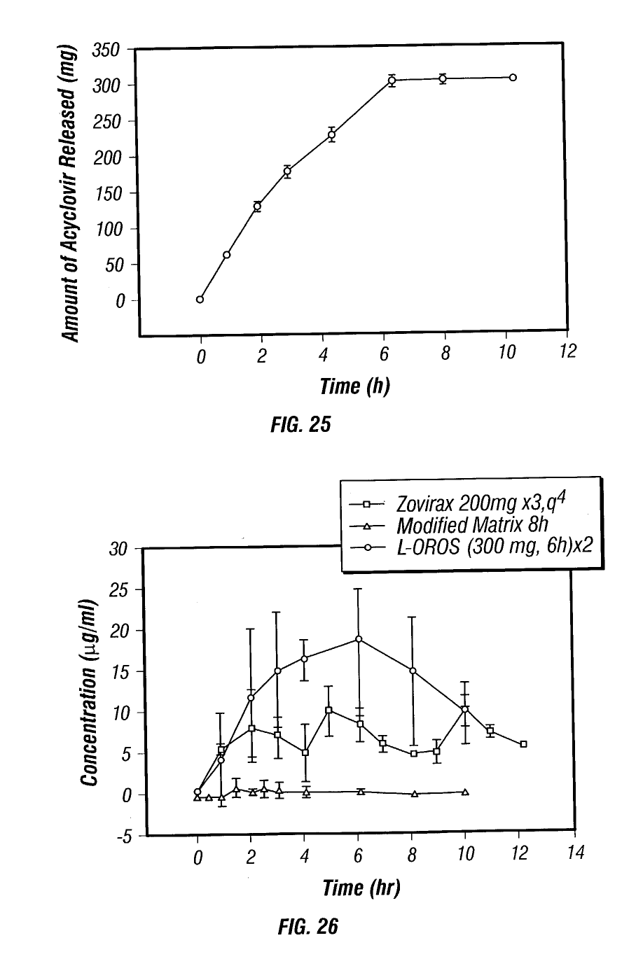

The release profile of a dosage form prepared according to this example was measured using a USP VII method in artificial intestinal fluid (AIF) without enzymes. As can be seen by reference to FIG. 25, 90% of the acyclovir contained in the dosage form was released over 6 hours (t₉₀=6 hours) at a constant rate.

### EXAMPLE 3

The dosage form described in Example 2 was tested in fasted mongrel dogs and compared to Zovirax (200 mg×3, tid, 4 h), a commercial acyclovir product, and to a modified matrix system having a t₉₀ of 8 hours. The modified matrix dosage form included acyclovir incorporated into a polymer matrix tablet having several insoluble bands coated on its surface. The modified matrix tablet swells extensively upon contact with gastric fluids, and the insoluble bands provided on the modified matrix tablet enabled the tablet to provide zero order drug release over 8 hours. The same set of mongrel dogs was used to obtain the bioavailability and plasma concentration data for each dosage form tested. In each instance, plasma samples were collected periodically, and the plasma concentration of acyclovir in the plasma samples collected was determined using HPLC. FIG. 26 provides a graph showing the plasma concentrations of acyclovir achieved using each of the various systems.

### EXAMPLE 4

A dosage form according to the present invention providing the controlled release of acyclovir over 10 hours was manufactured. To produce the dosage form, the manufacturing procedure detailed in Example 2 was generally followed, and the composition of the osmotic engine, the barrier layer, the formulation, and the semipermeable membrane of the dosage form were identical to those of dosage form described in Example 2. To achieve controlled release of the acyclovir formulation over 10 hours, however, the filled gelatin capsule described in Example 3 was sprayed with the semipermeable membrane coating solution until a semipermeable membrane weight of 65 mg was achieved. Such a dosage form will provide a to of 10 hours, with the acyclovir being released at a constant rate.

### EXAMPLE 5

The manufacturing procedure described in Example 2 is generally repeated to produce a dosage form according to the present invention containing a formulation including L-dopa and carbidopa as the therapeutic agents. The compositions of the osmotic engine, the barrier layer, and the rate controlling membrane are identical to those described in Example 2. In addition, the formulation included in the dosage form is homogeneously mixed and included in the gelatin capsule as described in Example 2. However, the formulation of the dosage form is composed of, in weight percent, 33.3% L-dopa, 8.3% Carbidopa, 14% lauric acid, and 44.4% Cremophor EL. Such a dosage form will provide a t₉₀ of approximately 6 hours, with the L-dopa and carbidopa being released at a constant rate.

### EXAMPLE 6

A dosage form according to the present invention providing the controlled release of L-dopa and carbidopa over 10 hours was manufactured. To produce the dosage form, the manufacturing procedure described in Example 5 was generally followed, and the composition of the osmotic engine, the barrier layer, the formulation, and the semipermeable membrane of the dosage form were identical to those of dosage form described in Example 5. To achieve controlled release of the acyclovir formulation over 10 hours, however, the filled gelatin capsule was sprayed with the semipermeable membrane coating solution until a semipermeable membrane weight of 65 mg was achieved. Such a dosage form will provide a t₉₀ of 10 hours, with the L-dopa and carbidopa being released at a constant rate.

We claim:

1. A formulation for the controlled delivery of a therapeutic agent, the formulation comprising a therapeutic agent exhibiting greater absorption in the upper gastrointestinal tract than in the lower gastrointestinal tract, a permeation enhancer, and a carrier capable of forming a bioadhesive gel, the formulation being prepared such that the formulation is released within the gastrointestinal tract as a liquid and forms a bioadhesive gel after a period of time

2. The formulation of claim 1, wherein the therapeutic agent is selected from a group consisting of acyclovir, gancyclovir, L-dopa, carbidopa, ABT-232, and metformin hydrochloride.

3. The formulation of claim 1, wherein the permeation enhancer is selected from a group consisting of ethylene-diamine tetra-acetic acid (EDTA), bile salt permeation enhancers, fatty acid permeation enhancers, acyl carnitines, and salicylates.

4. The formulation of claim 1, wherein the carrier comprises a nonionic surfactant.

5. The formulation of claim 4, wherein the nonionic surfactant is selected from a group consisting of Cremophor EL, Cremophor RH, Incordas 30, polyoxyethylene 5 castor oil, polyethylene 9 castor oil, polyethylene 15 castor oil, d-α-tocopheryl polyethylene glycol succinate (TPGS), myverol, oleth-3, oleth-5, polyoxyl 10 oleyl ether, oleth-20, steareth-2, stearteth-10, steareth-20, ceteareth-20, polyoxyl 20, cetostearyl ether, PPG-5 ceteth-20, PEG-6 capryl/capric triglyceride, Pluronic® L10, L31, L35, L42, L43, L44, L62, L61, L63, L72, L81, L101, L121, and L122, Tween 20, Tween 40, Tween 60, Tween 65, Tween 80, Tween 81, Tween 85, PEG 20 almond glycerides, PEG-60 almond glycerides, PEG-20 corn glycerides, and PEG-60 corn glycerides.

6. The formulation of claim 1, wherein the formulation further comprises a viscosity reducing agent.

7. The formulation of claim 6, wherein the viscosity reducing agent is selected from group consisting of polyoxyethylene 5 castor oil, polyoxyethylene 9 castor oil, labratil, labrasol, capmul GMO (glyceryl mono oleate), capmul MCM (medium chain mono- and diglyceride), capmul MCM C8 (glyceryl mono caprylate), capmul MCM C10 (glyceryl mono caprate), capmul GMS-50 (glyceryl mono stearate), caplex 100 (propylene glycol didecanoate), caplex 200 (propylene glycol dicaprylate/dicaprate), caplex 800 (propylene glycol di 2-ethyl hexanoate), captex 300 (glyceryl tricapryl/caprate), captex 1000 (glyceryl tricaprate), captex 822 (glyceryl triandecanoate), captex 350 (glyceryl tricaprylate/caprate/laurate), caplex 810 (glyceryl tricaprylate/caprate/linoleate), capmul PG8 (propylene mono caprylate), propylene glycol, and propylene glycol laurate (PGL).

8. The formulation of claim 1, wherein the formulation further comprises an antioxidant.

9. The formulation of claim 8, wherein the antioxidant is selected from a group consisting of butylated hydroxytoluene, ascorbic acid, fumaric acid, malic acid, ∝-tocopherol, ascorbic acid palmitate, butylated hydroxyanisole, propyl gallate, sodium ascorbate, and sodium metabisulfate.

10. A formulation for the controlled release of a therapeutic agent, the formulation comprising a therapeutic agent exhibiting greater absorption in the upper gastrointestinal tract than in the lower gastrointestinal tract, a permeation enhancer, and a carrier capable of forming a bioadhesive gel, wherein the therapeutic agent comprises between about 0.01 wt % and about 50 wt % of the formulation, the permeation enhancer comprises between about 11% and about 30% of the formulation, and the carrier comprising between about 35% and 88% of the formulation.

11. The formulation of claim 10, wherein the therapeutic agent, the permeation enhancer, and the carrier are included in amounts that allow the formulation to be released within the gastrointestinal tract as a liquid before forming a bioadhesive gel in-situ after a period of time.

12. A dosage form comprising:
a formulation comprising a hydrophilic macromolecule, a permeation enhancer, and a carrier capable of forming a bioadhesive gel, the formulation being formulated such that the formulation is released within the gastrointestinal tract as a liquid and forms a bioadhesive gel in-situ after the formulation has had some opportunity to spread across a surface of a gastrointestinal mucosal membrane; and
a delivery device configured to release the formulation within the gastrointestinal tract of a subject at a controlled rate over a period of time.

13. The dosage form of claim 16, wherein the delivery device comprises:
a gelatin capsule;
a deformable barrier layer formed on the gelatin capsule;
an osmotic layer formed on the barrier layer; and
a semipermeable membrane formed over the semipermeable membrane.

14. The dosage form of claim 16, wherein the delivery device comprises:
a capsule having an interior compartment, the interior compartment containing the formulation, an osmotic engine, and a barrier layer positioned between the formulation and the osmotic engine; and
a semipermeable membrane.

15. A dosage form providing controlled release of a therapeutic agent, the dosage form comprising:
a formulation including a therapeutic agent having a relatively higher absorption in an upper portion of a gastrointestinal tract of a subject than in a lower portion of the gastrointestinal tract of the subject, the formulation providing increased absorption of the therapeutic agent in the lower portion of the gastrointestinal tract; and
a delivery device configured to deliver the formulation over a prolonged period of time.
